(19) United States
(12) Patent Application Publication
(10) Pub. No.: US 2022/0202764 A1
(43) Pub. Date: Jun. 30, 2022

(54) PHARMACEUTICAL, PHYTO-CANNABINOID BASED COMPOSITIONS

(71) Applicants: AKASHA CORPORATION, Anguilla (AI); LIV INNOVATION SA, Lugano (CH)

(72) Inventor: Ronald Johannes GLAS, Lugano (CH)

(21) Appl. No.: 17/432,849

(22) PCT Filed: Feb. 21, 2020
(86) PCT No.: PCT/NL2020/050111

§ 371 Date: Aug. 20, 2021

(30) Foreign Application Priority Data
Feb. 21, 2019 (NL) 2022614

(51) Int. Cl. A61K 31/352; A61K 31/05

(52) U.S. Cl. CPC A61K 31/352; A61K 31/05

(57) ABSTRACT

The present invention relates to a pharmaceutical composition comprising a combination of the cannabinoids tetrahydrocannabivarin (THCV), cannabigerol (CBG), cannabichromene (CBC) and tetrahydrocannabinol (THC) for use in the prevention or treatment of disorders related to depression, fatigue, deteriorated alertness, excessive daytime sleepiness and reduced appetite, wherein the relative weight ratios of THCV:CBG:CBC:THC are between 1-10:1-10:1-10:1-10, and to a method of treating a mammal in need thereof, said mammal suffering from a disorder related to depression, fatigue, deteriorated alertness, excessive daytime sleepiness and reduced appetite, wherein a combination of an effective amount of cannabinoids selected from the group consisting of tetrahdrocannabivarin (THCV), cannabigerol (CBG), cannabichromene (CBC) and tetrahydrocannabinol (THC), is administered to said mammal, wherein the relative weight ratios of THCV:CBG:CBC:THC are between 1-10:1-10:1-10:1-10.

### FIELD OF THE INVENTION

The present invention relates to pharmaceutical, phyto-cannabinoid based compositions for use in the prevention or treatment of disorders related to depression, fatigue, deteriorated alertness, excessive daytime sleepiness and reduced appetite. The pharmaceutical, phyto-cannabinoid based compositions according to the invention find application as a mood enhancer, as a mental activator, as an energizer and as an appetite stimulator. The present invention also relates to methods for treating a mammal in need thereof, said mammal suffering from disorders related to depression, fatigue, deteriorated alertness, excessive daytime sleepiness and reduced appetite, wherein a combination of an effective amount of cannabinoids is administered to said mammal. The present invention also relates to a method of selecting a specific strain of Cannabis sativa having a cannabinoid profile comprising THCV, CBG, CBC and THC wherein the weight ratios of THCV:CBG:CBC:THC are between 1-10:1-10:1-10:1-10

### BACKGROUND OF THE INVENTION

Cannabinoids are organic compounds that are exclusively found in Cannabis sativa, ruderalis, indica strains and their blends. Cannabis sativa is the natural source of a set of at least 66 oxygen-containing aromatic hydrocarbon compounds that are known collectively as phytocannabinoids (ElSohly, M. A., “Chemical constituents of Cannabis, In: Grotenhermen, F. and Russo, E., editors. Cannabis and cannabinoids: Pharmacology, Toxicology and Therapeutic Potential”. Binghamton (NY): Haworth Press, 2002: 27-36). The n-propyl analogue of Δ-9 tetrahydrocannabinol (THC) which was first detected in Cannabis by Gill et al. (Gill, E. W., Paton, W. D. M., Pertweee, R. G., “Preliminary Experiments on the chemistry and pharmacology of Cannabis”, Nature 228, 134-136, 1970) and named Δ-9-tetrahydrocannabivarin (THCV) by Merkus (Merkus, F. W. H. M., “Cannabivarin and tetrahydrocannabivarin, two new constituents of hashish”, Nature 232, 579-580, 1971).

Over the past 50 years, a considerable research in medicinal chemistry has been carried out around the natural constituents of Cannabis sativa L. Following the identification of Δ-9 tetrahydrocannabinol (Δ-9 THC, (-)-(6aR,10aR)-6,6,9-trimethyl-3-pentyl-6a,7,8,10a-tetrahydro-6H-benzo[c]chromen-1-ol, CAS No. 1972-08-3) in 1964, critical chemical modifications, e.g. variation of the side chain at C(3) and the opening of the tricyclic scaffold, have led to the characterisation of potent and cannabinoid receptor subtype-selective ligands. Those ligands that demonstrate high affinity for the cannabinoid receptors and good biological efficacy are still used as powerful pharmacological tools.

Cannabinoids are from pharmaceutical and toxicological point of view the safest group of drugs that ever exist and show the longest long-term safety history of at least 4000 Years as well the longest scientific life cycle of any other drug.

The commercially available eurogeneric drug Modafinil (Modiodal®) is a modern medicine with medication and applications close related to the present invention. It is manufactured by the pharmaceutical company Cephalon Inc. and it is generally prescribed to treat narcolepsy. Modafinil is also described as a “wakefulness promoting agent” and is sometimes prescribed off-label for Attention-Deficit Hyperactivity Disorder (ADHD). Cephalon Inc. also markets the drug for improving “alertness” and reducing excessive daytime sleepiness, so that it will keep you alert without the twitchy side effects and potential for addiction of traditional stimulants. Modafinil has, however, side-effects including headache, nausea, nervousness, rhinitis, diarrhoea, back pain, anxiety, insomnia, dizziness and dyspepsia.

GB 2377633 A of GW Pharma Ltd. discloses pharmaceutical compositions comprising cannabinoids having specific ratios of cannabidiol (CBD) to tetrahydrocannabinol (THC), wherein the CBD is present in an amount greater than the amount of THC. The compositions are clinically useful in the treatment or management of specific diseases or medical conditions including inflammatory diseases, diseases or conditions wherein oxidative stress plays a role, psychotic disorders, epilepsy, movement disorders, stroke, head injuries, diseases which require appetite suppression, multiple sclerosis, spinal cord injury, peripheral neuropathy, cancer pain and migraine. The pharmaceutical compositions may further comprise THCV (tetrahydrocannabinovarin) and

CBDV (cannabidivarin). GB 2381194 A of GW Pharmaceuticals Ltd. discloses pharmaceutical formulations for use in the administration of medicaments, in particular lipophilic medicaments, via mucosal surfaces.

GB 2414933 A of GW Pharma Ltd. discloses the use of a combination of cannabinoids for the treatment of pain, inflammation and/or disease modification in arthritis. The cannabinoids are selected from CBD or cannabidivarin (CBDV) and THC or tetrahydrocannabinovarin (THCV) and are in a predefined ratio by weight of less than or equal to 19:1 of CBD or CBDV to THC or THCV.

GB 2432312 A of GW Pharma Ltd. discloses the use of a combination of cannabinoids in the treatment of neuropathic pain, in particular peripheral neuropathic pain. A combination of CBD and THC may be used, wherein the ratio of CBD:THC by weight is between 10:1 and 1:10.

However, there is still a need in the art for improved pharmaceutical compositions based on cannabinioids that are suitable to alleviate certain conditions or that are suitable for preventing or treating certain disorders.

### SUMMARY OF THE INVENTION

The present invention relates to a pharmaceutical composition comprising a combination of the cannabinoids tetrahydrocannabivarin (THCV), cannabigerol (CBG), cannabichromene (CBC) and tetrahydrocannabinol (THC) for use in the prevention or treatment of disorders related to depression, fatigue, deteriorated alertness, excessive daytime sleepiness and reduced appetite, wherein the relative weight ratios of THCV : CBG:CBC:THC are between 1-10:1-10:1-10:1-10 (in respective order).

The present invention further relates to methods for treating a mammal from in need thereof, said mammal suffering from disorders related to depression, fatigue, deteriorated alertness, excessive daytime sleepiness and reduced appetite, wherein a combination of an effective amount of cannabinoids is administered to said mammal, wherein in said combination the relative weight ratios of THCV:CBG:CBC:THC are between 1-10:1-10:1-10:1-10 (in respective order).The present invention further relates to the use of a pharmaceutical composition comprising a combination of the cannabinoids tetrahydrocannabivarin (THCV), cannabigerol (CBG), cannabichromene (CBC) and tetrahydrocannabinol (THC), wherein the relative weight ratios of THCV:CBG:CBC:THC are between 1-10:1-10:1-10:1-10 (in respective order), as a mood enhancer, as a mental activator, as an energizer, as an appetite stimulator or a combination of any of these uses. The preference relative weight ratio of THCV, CBG and CBC are 1:1:1.

The present invention relates to a pharmaceutical composition comprising a combination of the cannabinoids tetrahydrocannabivarin (THCV), cannabigerol (CBG), cannabichromene (CBC) and tetrahydrocannabinol (THC) for use in the prevention or treatment of disorders related to depression, fatigue, deteriorated alertness, excessive daytime sleepiness and reduced appetite, wherein the relative weight ratios of THCV:CBG:CBC:THC are between 1-10:1-10:1-10:1 10, and to a method of treating a mammal in need thereof, said mammal suffering from a disorder related to depression, fatigue, deteriorated alertness, excessive daytime sleepiness and reduced appetite, wherein a combination of an effective amount of cannabinoids selected from the group consisting of tetrahydrocannabivarin (THCV), cannabigerol (CBG), cannabichromene (CBC) and tetrahydrocannabinol (THC), is administered to said mammal, wherein the relative weight ratios of THCV:CBG:CBC:THC are between 1-10:1-10:1-10:1-10.

The present invention also relates to a method of selecting a specific strain of Cannabis sativa having a cannabinoid profile comprising THCV, CBG, CBC and THC wherein the weight ratios of THCV:CBG:CBC:THC are between 1-10:1-10:1-10:1-10.

### DESCRIPTION OF THE DRAWINGS

FIG. 1 shows the chemotypes and their cannabinoid profiles.

### DETAILED DESCRIPTION OF THE INVENTION

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the element are present, unless the context clearly requires that there is one and only one of the elements. The indefinite article “a” or “an” thus usually means “at least one”.

Tetrahydrocannabivarin (THCV) is also known as 6,6,9-trimethyl-3-propyl-6a,7,8,10a-tetrahydrobenzo[c]chromen-1-ol (CAS. No. 28172-17-0). Tetrahydrocannabinol (THC) is also known as (-)-(6aR,10aR)-6,6,9-trimethyl-3-pentyl-6a,7,8,10a-tetrahydro-6H-benzo[c]chromen-1-ol (CAS. No. 1972-08-3). No CAS No. is known for cannabichromene. In this document, the term THC also includes 9′-or 8′-carboxylated analogues (e.g. carboxylic acids and carboxylic acid esters) thereof, e.g. 11-nor-9-carboxy-Δ9-tetrahydrocannabinol(1-hydroxy-6,6-dimethyl-3-pentyl-6a,7,8, 10a-tetrahydrobenzo[c]-chromene-9-carboxylic acid; Cas. No. 64280-14-4).

Cannabigerol (CBG) is also known as (E)-2-(3,7-Dimethylocta-2,6-dienyl)-5-pentylbenzene-1,3-diol (CAS. Nos. [2808-33-5], [25654-31-3] (E); [9500-1-70-0] undefined configuration. Isomers:[25654-32-4] (Z)). CBG-analogues according to Formula 1 are listed in Table 1.

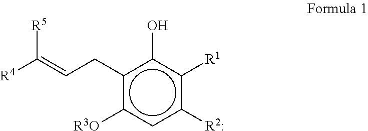

TABLE 1
CBG-analogues according to Formula 1

| Compound | Cis/Trans | R¹ | R² | R³ | R⁴ | R⁵ |
|---|---|---|---|---|---|---|
| Cannabigerolic | cis | COOH | n-C₅H₁₁ | H | (CH₂)₂CH═C(CH₃)₂ | OH |
| acid A | | | | | | |
| [(E)-CBGA-C₅ | | | | | | |
| A] | | | | | | |
| Cannabigerolic | cis | COOH | n-C₅H₁₁ | Me | (CH₃)₂CH═C(CH₃)₂ | Me |
| acid A | | | | | | |
| monomethyl | | | | | | |
| ether | | | | | | |
| [(E)-CBGAM-C₅ | | | | | | |
| A] | | | | | | |
| Cannabigerol | cis | H | n-C₅H₁₁ | H | (CH₃)₂CH═C(CH₃)₂ | OH |
| [(E)-CBG-C₅] | | | | | | |
| Cannabigerol | cis | H | n-C₅H₁₁ | Me | (CH₂)₂CH═C(CH₃)₂ | Me |
| monomethyl | | | | | | |
| ether | | | | | | |
| [(E)-CBGM-C₅] | | | | | | |
| Cannabigerovarinic | cis | COOH | n-C₃H₇ | H | (CH₃)₂CH═C(CH₃)₂ | OH |
| acid A | | | | | | |
| [(E)-CBGVA-C₃ | | | | | | |
| A] | | | | | | |
| Cannabigerovarin | cis | H | n-C₃H₇ | H | (CH₂)₂CH═C(CH₃)₂ | OH |
| [(E)-CBGV-C₃] | | | | | | |
| Cannabinerolic | trans | COOH | n-C₅H₁₁ | H | Me | (CH₂)₂CH═C(CH₃)₂ |
| acid A | | | | | | |
| [(Z)-CBGA-C₅ | | | | | | |
| A] | | | | | | |

Cannabichromene (CBC) and its analogues have the formula according to Formula 3.

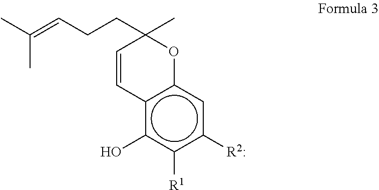

| | R¹ | R² |
|---|---|---|
| (±)-cannabichromene | H | n-C₅H₁₁ |
| (±)-cannabichromenic acid | COOH | n-C₅H₁₁ |
| (±)-cannabivarichromene | H | n-C₃H₇ |
| (±)-cannabichromevarinic acid | COOH | n-C₃H₇ |

Hence, in this document, the term e.g. “CBG” is to be understood as representing the group of compounds CBG and CBG-analogues as is shown above in Formula 1 and Table 1. As will be apparent to the person skilled in the art, the term “CBC” have a similar meaning, i.e. that it represents the group of compounds CBC and its analogues as is shown above.

Besides disorders mentioned above, the pharmaceutical compositions according to the present invention and/or the combinations of cannabinoids according to the present invention is also effective as a mood enhancer and/or a stimulant, relieves fatigue, improves “alertness”, and is suitable for the treatment of depression.

The pharmaceutical compositions according to the present invention and/or the combinations of cannabinoids according to the present invention are also suitable for the prevention or treatment of conditions selected from narcolepsy, a disorder marked by uncontrollable attacks of daytime sleepiness, Alzheimer's disease, attention-deficit disorder (ADHD), myotonic dystrophy, multiple sclerosis-induced fatigue, post-anaesthesia grogginess, cognitive impairment in schizophrenia, spasticity associated with cerebral palsy, age-related memory decline, idiopathic hypersomnia, methamphetamine (‘Ice’) abuse, apathy in the elderly, jet lag, cancer-associated fatigue and opioid-induced sedation, fatigue in Charcot-Marie-Tooth Disease (CMT), everyday cat-napping, chronic fatigue and languid and listless modes.

The pharmaceutical compositions according to the present invention and/or the combinations of cannabinoids according to the present invention are also effective in the treatment of “atypical” depression. Atypical depression is marked by hypersomnia, hyperphagia (over-eating), low energy, and rejection-sensitivity. The syndrome is actually quite common.

The pharmaceutical compositions according to the present invention and/or the combinations of cannabinoids according to the present invention are also effective as mood-brightening psycho-stimulating agents, i.e. they enhance wakefulness and vigilance, although their pharmacological profiles are notably different from amphetamines, methylphenidate (Ritalin®) and cocaine.

Accordingly, the pharmaceutical compositions according to the present invention and/or the combinations of cannabinoids according to the present invention can be used for relieving fatigue and for energizing, as anti-depression agents, activating agents, anti- sleep agents, as mood enhancers and as stimulants. The compositions can further be used as a wakefulness promoting agent', a stimulant, improving alertness and reducing excessive daytime sleepiness. It reduces excessive somnolence and enhances mood in patients.

The present invention therefore relates to the use of a combination of the canabinoids tetrahydrocannabivarin (THCV), cannabigerol (CBG), cannabichromene (CBC) and tetrahydrocannabinol (THC) and pharmaceutical compositions for use in the prevention or treatment of in the prevention or treatment of disorders related to depression, fatigue, deteriorated alertness, excessive daytime sleepiness and reduced appetite, wherein the relative weight ratios of THCV:CBG:CBC:THC are between 1-10:1-10:1-10:1-10. Most preferably, the relative weight ratio is 1:1:1:1-10.

In an embodiment of the present invention, the composition is for use in the prevention or treatment of disorders related to depression, fatigue, deteriorated alertness, and excessive daytime sleepiness. In another embodiment, the composition is for use in the prevention or treatment of disorders related to depression, deteriorated alertness, excessive daytime sleepiness and reduced appetite. In preferred embodiment, the composition is for use in the prevention or treatment of disorders related to depression. According to the invention, it is preferred that the disorder is selected from the group consisting of narcolepsy, daytime sleepiness, Alzheimer's disease, depression, attention-deficit disorder (ADHD), myotonic dystrophy, post-anaesthesia grogginess, cognitive impairment in schizophrenia, spasticity associated with cerebral palsy, age- related memory decline, idiopathic hypersomnia and methamphetamine (‘Ice’) abuse.

Preferably, in the pharmaceutical compositions according to the present invention, the relative weight ratios of THCV:CBG:CBC:THC are 1-5:1-5:1-5:1 10, more preferably 1:1:1:1-5.

It is preferred that when the disorder is (chronic) depression, the relative weight ratios of THCV:CBG:CBC:THC are 1-4:1-4:1-4:1 10, more preferably

Preferably, the pharmaceutical compositions comprise a unit dosage form comprising 1-12 mg of each cannabinoid THCV, CBG, CBC and THC. Unit dose ranges are preferably in the range of between 1 and 10 mg of each cannabinoid THCV, CBG, CBC and THC.

The maximum daily dosage is preferably less than or equal to 120 mg of THCV, CBG and CBC. The maximum daily dosage is preferably less than or equal to 130 mg of

THC, more preferably less than or equal to 120 mg.

According to the present invention, the pharmaceutical compositions are in a form selected from the group consisting of gels, gel sprays, tablets, liquids, capsules, compositions for vaporization and compositions for nebulisation.

As will be apparent to the person skilled in the art, one or more of THCV, CBG, CBC and THC may be synthetic. However, it is preferred that they are obtained from natural sources.

Optionally, the pharmaceutical composition according to the present invention may comprise one or more other drugs, preferably one or more sleep inducing drugs or sedatives.

The present invention also relates to a method of treating a mammal in need thereof, said mammal suffering from a disorder related to depression, fatigue, deteriorated alertness, excessive daytime sleepiness and reduced appetite, wherein a combination of an effective amount of cannabinoids selected from the group consisting of tetrahydrocannabivarin (THCV), cannabigerol (CBG), cannabichromene (CBC) and tetrahydrocannabinol (THC), is administered to said mammal, wherein the relative weight ratios of THCV:CBG:CBC:THC are between 1-10:1-10:1-10:1-10.

Preferably, THC is administered separately, simultaneously or sequentially to CB THCV, CBG and CBC.

The invention also relates to the use of a combination of the cannabinoids THCV, CBG, CBC and THC for the use as a stimulant and can be described as a wakefulness promoting agent. The combination and/or pharmaceutical composition improves alertness and reduces excessive daytime sleepiness. It provokes an energising, activating state of mind and uppers physical behaviour; for the treatment of chronic fatigue, against languid and listless modes. It also reduces excessive somnolence and enhances mood in patients. Further advantages of the combination and/or composition according to the invention include a stimulation of physical behaviour, of sensory systems such as sight, hearing, seeing, and of the mind. The combination and/or composition according to the invention also improve excessive daytime somnolence and fatigue in primary biliary cirrhosis.

The pharmaceutical compositions and/or combinations according to the present invention may comprise one or more other cannabinoid or non-cannabinoid based, active ingredients selected from the group consisting of the following compounds (named “cryptic” spots on a TLC plate as obtained in the method disclosed in US 2007077660, incorporated by reference):

1. plastids (xanthophylls like);

2. plant pigments (chlorophyll A and B, xanthophylls and phaeophytin);

3. components of the plastids such as membranes (thylakolid) and proteins.

These other cannabinoid or non-cannabinoid based, active ingredients are preferably present in the pharmaceutical composition according to the present invention in amount of 0.01-12 wt. %, based on the total weight of the pharmaceutical composition.

Pharmaceutical compositions according to the present invention comprising CBG and THC can be used for the treatment of depression and depressive moods, wherein the weight ratio of CBG:THC is between 10:1 and 1:10, more preferably between 1:5 and 5:1. These compositions also comprise THCV and CBC in weight ratios of 10:1 to 1:10, preferably 5:1 to 1:5.

Pharmaceutical compositions according to the present invention comprising CBG and THC can be used as mood stabiliser and mood relaxator, wherein the ratio of CBG:THC by weight is preferably between 7:3 and 3:7, more preferably between 2:5 and 5:2. These compositions also comprise THCV and CBC in weight ratios of 10:1 to 1:10, preferably 5:2 to 2:5, and more preferably 1:1.

Preferably, the pharmaceutical compositions according to the present invention are packaged for delivery in a titratable dosage form. The term “titratable” as used herein is defined as meaning that the patient is provided with a medication that is in such a form that smaller doses than the unit dose can be taken.

Titration of doses in a patient related manner, are beneficial to the patient as they are able to take smaller of doses of the medication until the drug is efficacious. It is understandable that not all patients will require exactly the same dose of medication, for example patients of a larger build or faster metabolism.

Preferably the maximum daily dosage dose of medicament is less than or equal to 120 mg in a 1:1:1 formulation of THCV, CBG and CBC oil and less than or equal to 130 mg

THC and 130 mg of the formulation from the art and less than or equal to 30 mg of THC.

Preferably the cannabinoids are packaged for delivery such that delivery is targeted to an area selected from one or more of the following: sublingual, buccal, oral, rectal, nasal, and the pulmonary system as vapour, intravenous, intra-arterial, topical, by injection, intraperitoneal, intrapleural, orally, subcutaneously, intramuscularly, intra-epidermal. Illustrative methods of administration are vapour, oral and intravenous. The oral formulations may be solutions, suspensions, suppositories, tablets, granules, powders, capsules, ointments, or creams. The intravenous formulations may be solutions or suspensions, including compositions comprising liposomes.

In the preparation of the pharmaceutical compositions, a solvent (e.g. water or physiological saline), a solubilising agent (e.g. ethanol, Polysorbates, or Cremophor EL7), an isotonising agent, a preservative, an antioxidant, an excepient (e.g. lactose, starch, crystalline cellulose, mannitol, maltose, calcium hydrogen phosphate, light silicic acid anhydride or calcium carbonate), a binder (e.g. polivinylpyrrolidone, hydroxypropyl cellulose, ethyl cellulose, carboxymethyl cellulose or gum Arabic), a lubricant (e.g., magnesium stearate, talc or a hardened oil), or a stabilizer may be added. If necessary, glycerine, dimethylacetarnide, 70% sodium lactate, a surfactant, or a basic substance such as sodium hydroxide, ethylenediamine, ethanolamine, sodium bicarbonate, arginine, meglumine, or trisaminomethane is added. Pharmaceutical preparations such as solutions, tablets, granules or capsules can be formed with these components. Compositions for slow or controlled release can for example be manufactured according to the method disclosed in U.S. Pat. No. 4,880,830.

Generally, the oral pharmaceutical compositions of the present invention provide a daily dosage of about 11 mg to about 200 mg, preferably about 10 mg to about 100 mg per day, even more preferably about 20 mg to about 60 mg of total cannabinoid components, which compositions are administered about 1, 2, 3, 4 or 5 times per day.

Additionally the pharmaceutical compositions may further comprise one or more carrier solvents. Preferably the carrier solvents are ethanol and/or propylene glycol. More preferably, the ratio of ethanol to propylene and glycol is between 4:1 and 1:4. More preferably still the ratio is about 1:1.

Preferably, the combinations of cannabinoids are present as a Cannabis based medicine ice cold water powder extract of the dried flower tips.

Preferably, the combination of cannabinoids comprises:

a Cannabis based medicinal extract which comprises cannabigerol (CBG) and cannabinodiol (CBND) in a 1:1 ratio by weight at more than 90% of the of the total cannabinoid content in the extract; and

a Cannabis based medicinal extract which comprises THC and CBND at more than 90% of the total cannabinoid content in the extract.

In an embodiment the composition further comprises cannabinodiol (CBND), preferably in a ratio by weight CBG:CBND of 1:1.

When CBND is added, it functions as catalyst and enhances the effect of THC with CBG.

The combination of cannabinoids and/or the pharmaceutical compositions according to the present invention can also be used for other purposes, in particular in the treatment of the treatment of jet-lag, multiple sclerosis-induced fatigue, cancer-associated fatigue and opioid-induced sedation, fatigue in Charcot-Marie-Tooth Disease (CMT), everyday cat-napping, chronic fatigue and against a languid and listless mood. It relieves fatigue in palliative care patients, wherein the relative weight ratios of THCV:CBG:CBC:THC is preferably between 1-10:1-10:1-10:1 10, preferably 1-5:1-5:1-5:1-10, more preferably 1:1:1:1 to 5.

The combination of cannabinoids and/or the pharmaceutical compositions according to the present invention can also be used as a stimulant or as a wakefulness promoting agent since it enhances wakefulness and vigilance, alertness and reduces excessive daytime sleepiness. The combination and/or composition have mood-brightening psycho-stimulating properties.

Examples of pharmaceutical compositions according to the present invention are a Cannabis based medicinal extract which comprises THCV, CBG, CBC and THC in the preferred 1:1:1:1-5 weight ratio, wherein THCV, CBG, CBC and THC form more than 90 wt. % of the total cannabinoid content in the extract, based on the total weight of the extract. In other words, the compositions comprise less than 10 wt. % of cannabinoid compounds other than THCV, CBG, CBC, and THC.

In an embodiment, the pharmaceutical composition comprises no CBDV. In an embodiment, the pharmaceutical composition comprises no CBDVA. In an embodiment, the pharmaceutical composition comprises no CBDA. In an embodiment, the pharmaceutical composition comprises no CBDV, CBDVA, and CBDA.

In another aspect, the invention relates to a method of selecting a specific strain of Cannabis sativa having a cannabinoid profile comprising THCV, CBG, CBC and THC wherein the weight ratios of THCV:CBG:CBC:THC are between 1-10:1-10:1-10:1-10, preferably 1-5:1-5:1-5:1 10, more preferably 1:1:1:1 5 or 1-4:1-4:1-4:1-10. In the extract of the selected strain, the combined amount of THCV, CBG, CBC and THC is preferably at least 90 wt. % of the total cannabinoid content.

Identification of the present cannabinoids can be done in the following manner: By means of a thin layer chromatography (TLC), as described in US20070077660A1. “TLC” as used in the present description stands for thin-layer chromatography. This is a chromatography technique used to separate non-volatile mixtures. Thin-layer chromatography is performed on a sheet of glass, plastic, or aluminium foil, which is coated with a thin layer of adsorbent material, usually silica gel, aluminium oxide (alumina), or cellulose. This layer of adsorbent is known as the stationary phase. After the sample has been applied on the plate, a solvent or solvent mixture (known as the mobile phase) is drawn up the plate via capillary action. Because different analytes ascend the TLC plate at different rates, separation is achieved.

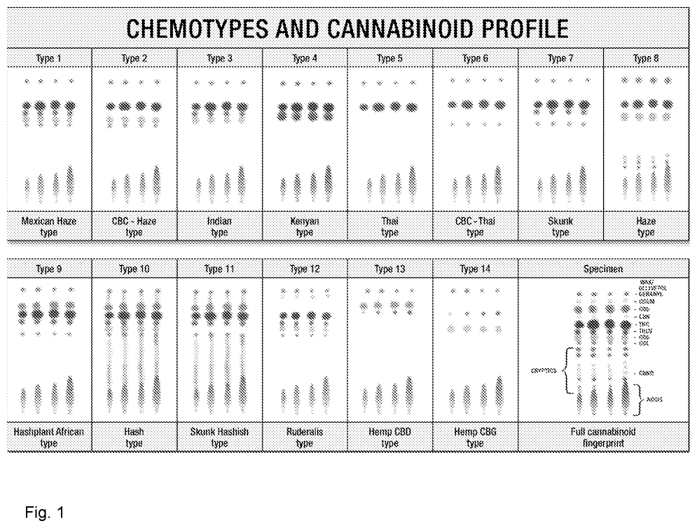

FIG. 1 shows the profiles for the different chemotypes. It can be seen from FIG. 1 that for the present invention that type 10 and type 12 are of main interest because of the present or absent cannabinoids.

### EXAMPLE

### Method of Preparation of Pharmaceutical Composition

Step 1: Chopping to predominantly 2-3 mm.
Step 2: Decarboxylate at 100-150° C. to form neutral forms.
Step 3: Extraction with a specific volume of Ethanol or liquid carbon.
Step 4: Removal by film-rotavoparization or depressuration of CO₂ resp.
Step 5: Winterisation:Winterisation removes unwanted components from the crude extract. The first step is to dilute the crude extract in ethanol and store the mixture at the freezing point of ethanol (114.1 ° C.) for at least 24 hours. This prompts the removal of lipids and waxes from the extract.
Step 6: Removal of unwanted waxes by cold filtration. Filtration: To remove precipitates and other particulates from the extract, one can use vacuum filtration via a Buchner funnel or a plate press. The filter micron range should be 0.45 or less.
Step 7: Removal of ethanol from the filtrate by thin film evaporation under reduced pressure, closed loop distillation. Distillation:To produce a cannabinoid-rich distillate product, one can either short path distillation, fractional distillation or wiped film distillation.
Step 8: CPC:Separating and/or purifying cannabinoids, comprising at least one liquid-liquid partition chromatography step, or the use of a centrifugal distribution chromatograph for liquid-liquid partition chromatography to separate and/or purify cannabinoids using a solvent selected from cyclohexane, heptane, n-heptane, iso-heptane, octane, n-octane, iso-octane, which is kept stationary by centrifugal force and a second immiscible liquid phase can be pumped through as a mobile phase. More details can be found in WO2016135346A1. Fractionation of neutral cannabinoids by CPC using the two-phase system hexane/acetone/acetonitrile, 5:2:3 (v:v:v, solvent system 2). The CPC is operated in ascending mode, with the lower (acetonitrile-rich) phase used as stationary phase and the upper (hexane-rich) upper phase as mobile phase. Flow-rate set at 5 ml/min and rotation speed at 600 rpm. The volume of stationary phase at 65 ml. Dissolve the sample to a final volume of 5 ml of upper phase for injection. Fraction size are collected. Analyses of fractions by TLC and further analysis by HPLC. Resulting Fraction contains a high proportion (>90%, preferably >95%) of the desired compound. This method is described in Hazekamp et al., Preparative Isolation of Cannabinoids from Cannabis sativa by Centrifugal Partition Chromatography, Journal of Liquid Chromatography & Related Technologies 27(15):2421-2439 December 2004.
Step 9: One or more (desired) isolated cannabinoids are selected and combined to be present in the pharmaceutical composition. Preferably, no other cannabinoids than the selected one(s) are present in the composition; however, trace amounts may be present. Preferably, the selected cannabinoids in the pharmaceutical composition are present in the same respective weight ratios as in the Cannabis strain.

### Example 1

A cannabinoid composition was isolated and analysed from C. sativa Simplex strain # 54 by the method disclosed in US 2007077660. The following compounds with the following relative weights were identified:

THCV: 1.4 wt. %

CBG 1.5 wt. %

CBC: 1.3 wt. %

THC: 15 wt. %

A cannabinoid composition was prepared according to the 11 step method as described above, wherein the four mentioned cannabinoids were present in the same relative weight ratios as listed. No other cannabinoids were present in the composition.

The composition and individual components were evaluated on their pharmacological effects in a randomized, single-blind study (167 panel members) All panel members experienced an energizing and activating effect. All panel members took daily unit dosages of 100mg (a unit dosage comprises a total amount of THCV, CBG, CBC and THC of % to 12 mg). Panel members stopped intake of the daily dosage after experiencing a pronounced and adequate effect.

Adverse side effects were not reported, but a sense of well-being was frequently experienced and sometimes a dry mouth.

### Example 2

A composition was made consisting of THCV; CBG; CBC, and THC in a weight ratio of 1-5:1-5:1-5:1-10.

### Example 3

A composition was made consisting of THCV; CBG; CBC, and THC in a weight ratio of 1:10:1:10. The composition and individual components were evaluated on their pharmacological effects by 67 panel members. From the panel members, 20-30% experienced an energizing and activating effect, and 70-80% did not experience an energizing and activating effect. All panel members took daily unit dosages of 1-12 mg.

### Comparative Example 1

A composition was made consisting of THCV; CBG; and CBC. There was no THC present in this composition. THC is the psychoactive component which will influence the psyche of an individual therefore to accomplish a psychological effect which has influence on the state of mind and state of being.

### CLAUSES

1. Pharmaceutical composition comprising a combination of the cannabinoids tetrahydrocannabivarin (THCV), cannabigerol (CBG), cannabichromene (CBC) and tetrahydrocannabinol (THC) for use in the prevention or treatment of disorders related to depression, fatigue, deteriorated alertness, excessive daytime sleepiness and reduced appetite, wherein the relative weight ratios of THCV:CBG:CBC:THC are between 1-10:1-10:1-10:1-10.

2. Pharmaceutical composition according to clause 1, wherein the disorder is selected from the group consisting of narcolepsy, daytime sleepiness, Alzheimer's disease, depression, attention-deficit disorder (ADHD), myotonic dystrophy, post-anaesthesia grogginess, cognitive impairment in schizophrenia, spasticity associated with cerebral palsy, age-related memory decline, idiopathic hypersomnia and methamphetamine (‘Ice’) abuse.

3. Pharmaceutical composition according to clause 1, wherein the relative weight ratios of THCV:CBG:CBC:THC are 1-5:1-5:1-5:1-10.

4. Pharmaceutical composition according to clause 3, wherein the relative weight ratios of THCV:CBG:CBC:THC are 1:1:1:1-5.

5. Pharmaceutical composition according to clause 1, wherein the disorder is (chronic) depression and wherein the relative weight ratios of THCV:CBG:CBC:THC are 1-4:1-4:1-4:1-10.

6. Pharmaceutical composition according to clause 1, wherein the pharmaceutical composition comprises a unit dosage form comprising 1-12 mg of each cannabinoid THCV, CBG, CBC and THC.

7. Pharmaceutical composition according to clause 1, wherein the maximum daily dosage is less than or equal to 120 mg of THCV, CBG and CBC.

8. Pharmaceutical composition according to clause 7, wherein the maximum daily dosage is less than or equal to 130 mg of THC.

9. Pharmaceutical composition according to clause 1, wherein the pharmaceutical composition is in the form selected from the group consisting of gels, gel sprays, tablets, liquids, capsules, compositions for vaporization and compositions for nebulisation.

10. Pharmaceutical composition according to clause 1, wherein one or more of THCV, CBG, CBC and THC is synthetic.

11. Pharmaceutical composition according to clause 1, wherein the pharmaceutical composition comprises one or more other drugs.

12. A method of treating a mammal in need thereof, said mammal suffering from a disorder related to depression, fatigue, deteriorated alertness, excessive daytime sleepiness and reduced appetite, wherein a combination of an effective amount of cannabinoids selected from the group consisting of tetrahydrocannabivarin (THCV), cannabigerol (CBG), cannabichromene (CBC) and tetrahydrocannabinol (THC), is administered to said mammal, wherein the relative weight ratios of THCV:CBG:CBC:THC are between 1-10:1-10:1-10:1-10.

13. The method according to clause 12, wherein THC is administered separately, simultaneously or sequentially to CB THCV, CBG and CBC.

1. Pharmaceutical composition comprising a combination of the cannabinoids tetrahydrocannabivarin (THCV), cannabigerol (CBG), cannabichromene (CBC) and tetrahydrocannabinol (THC) for use in the prevention or treatment of disorders related to depression, fatigue, deteriorated alertness, excessive daytime sleepiness and reduced appetite, wherein the relative weight ratios of THCV:CBG:CBC:THC are between 1-10:1-10:1-10:1-10.

2. Pharmaceutical composition according to claim 1, wherein the disorder is selected from the group consisting of narcolepsy, daytime sleepiness, Alzheimer's disease, depression, attention-deficit disorder (ADHD), myotonic dystrophy, post-anaesthesia grogginess, cognitive impairment in schizophrenia, spasticity associated with cerebral palsy, age-related memory decline, idiopathic hypersomnia and methamphetamine (‘Ice’) abuse.

3. Pharmaceutical composition according to claim 1, wherein the relative weight ratios of THCV:CBG:CBC:THC are 1-5:1-5:1-5:1-10.

4. Pharmaceutical composition according to claim 3, wherein the relative weight ratios of THCV:CBG:CBC:THC are 1:1:1:1-5.

5. Pharmaceutical composition according to claim 1, wherein the disorder is (chronic) depression and wherein the relative weight ratios of THCV:CBG:CBC:THC are 1-4:1-4:1-4:1-10.

6. Pharmaceutical composition according to claim 1, wherein the pharmaceutical composition comprises a unit dosage form comprising between 1-12 mg of each of the cannabinoids THCV, CBG, CBC and THC.

7. Pharmaceutical composition according to claim 1, wherein the maximum daily dosage of THC is less than or equal to 130 mg.

8. Pharmaceutical composition according to claim 1, wherein the maximum daily dosage of each of the cannabinoids THCV, CBG, CBC and is less than or equal to 120 mg.

9. Pharmaceutical composition according to claim 1, wherein the pharmaceutical composition is in the form selected from the group consisting of gels, gel sprays, tablets, liquids, capsules, compositions for vaporization and compositions for nebulisation.

10. Pharmaceutical composition according to claim 1, wherein one or more of the cannabinoids THCV, CBG, CBC and THC is synthetic.

11. Pharmaceutical composition according to claim 1, wherein the pharmaceutical composition further comprises one or more drugs.

12. Pharmaceutical composition according to claim 1, wherein THC is to be administered separately, simultaneously or sequentially to THCV, CBG and CBC.

13. Pharmaceutical composition according to claim 1, wherein the pharmaceutical composition comprises no CBDV, CBDVA, and/or CBDA.

14. Pharmaceutical composition according to claim 1, wherein the composition further comprises cannabinodiol (CBND), preferably in a ratio by weight CBG:CBND of 1:1.
